(19) United States
(12) Patent Application Publication
(10) Pub. No.: US 2018/0092998 A1
(43) Pub. Date: Apr. 5, 2018

(54) COMPOSITIONS AND METHODS FOR DIAGNOSING AND TREATING MACROPHAGE-RELATED DISORDERS USING CARBOHYDRATE-BASED MACROMOLECULAR CARRIER

(71) Applicant: CARDINAL HEALTH 414, LLC, DUBLIN, OH (US)

(72) Inventor: Frederick O Cope, Dublin, OH (US)

(73) Assignee: CARDINAL HEALTH, 414 LLC, DUBLIN, OH (US)

(21) Appl. No.: 15/724,959

(22) Filed: Oct. 4, 2017

Related U.S. Application Data
(60) Provisional application No. 62/404,111, filed on Oct. 4, 2016.

(51) Int. Cl. A61K 51/04; A61P 29/00; A61P 35/00; A61P 31/18; A61P 25/00

(52) U.S. Cl. CPC A61K 51/0491; A61P 29/00; A61P 25/00; A61P 31/18; A61P 35/00

(57) ABSTRACT

A method of diagnosing an inflammasome-mediated disorder includes administering a pharmaceutical composition to a subject. The composition includes a carrier molecule having a detectable moiety attached thereto. The carrier molecule includes a non-toxic carbohydrate-based backbone. The method also includes after the administering step, detecting a presence of the detectable moiety at a predetermined location in the subject.

### BACKGROUND

Various receptor-binding compounds have been developed for use in the diagnosis or treatment of various medical conditions. Such receptor-binding compounds typically are designed to bind to one or more receptor sites on one or more specific proteins. Receptor-binding compounds can be used to deliver therapeutic or diagnostic agents to specific target cells, or even to block certain receptors for therapeutic reasons.

By way of example, U.S. Pat. No. 6,409,990 (“the '990 patent”), titled “Macromolecular Carrier for Drug and Diagnostic Agent Delivery,” which issued on Jun. 25, 2002 and is incorporated herein by way of reference, discloses receptor-binding macromolecules which have been shown to be useful as carrier molecules for the delivery of radioisotopes for use in sentinel node imaging for staging breast cancer and melanoma. The carrier molecules described in the '990 patent exhibit significant and sustained uptake by sentinel lymph nodes, thus allowing the delivery of the radioisotopes attached to the carrier molecule.

By way of a more specific example, one currently marketed diagnostic agent produced in accordance with the '990 patent is technetium Tc 99m tilmanocept, which is marketed under the name LYMPHOSEEK® Injection kit. The LYMPHOSEEK kit is distributed in the form of vials containing tilmanocept powder. The tilmanocept powder is radiolabeled with technetium Tc 99m prior to use in order to prepare the technetium Tc 99m tilmanocept diagnostic agent. This diagnostic agent is formed when a technetium Tc 99m pertechnetate solution is added to the vial containing the tilmanocept powder, such that the technetium Tc 99m binds to the diethylenetriaminepentaacetic acid (“DTPA”) moieties of the tilmanocept molecule. The resulting radioactive diagnostic agent is approved for use in the lymphatic mapping using a hand-held gamma counter in order to assist in the localization of lymph nodes draining a primary tumor site (i.e., sentinel lymph nodes) in patients having breast cancer or melanoma.

Tilmanocept, the non-radiolabeled precursor of the LYMPHOSEEK® diagnostic agent, has a dextran backbone to which a plurality of amino-terminated leashes (—O(CH₂)₃S(CH₂)₂NH₂) are attached. In addition, mannose moieties are conjugated to amino groups of some of the leashes, and the chelator diethylenetriamine pentaacetic acid (DTPA) is conjugated to the amino group of other leashes. Tilmanocept generally consists of dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio) ethyl]amino]ethyl]thio]propyl ether complexes, and generally has the following structure:

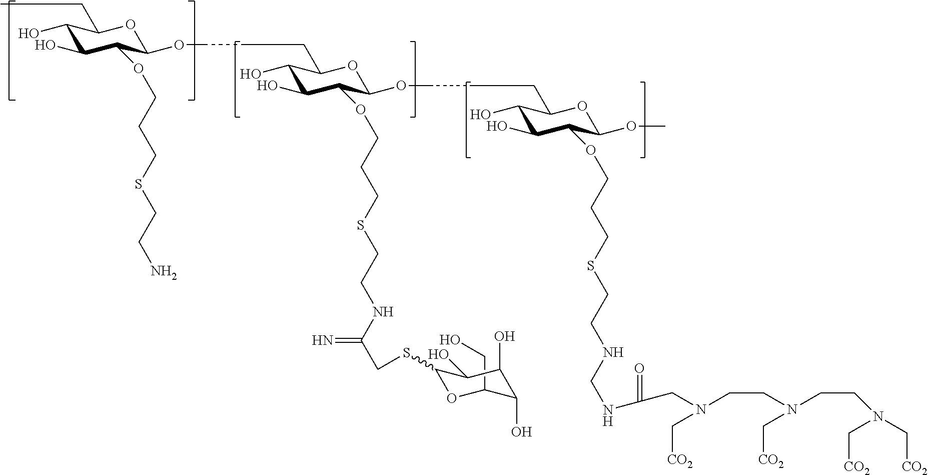

It should be noted that in some instances certain ones of the glucose moieties may have no attached aminothiol leash.

The DTPA chelator portion of tilmanocept is used for the attachment of the radioactive isotope Tc 99m to the carrier molecule. After radiolabeling (e.g., as described in the '990 patent), technetium tilmanocept is formed: technetium Tc 99m, dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio) ethyl]amino]ethyl]thio]propyl ether complexes. Technetium Tc 99m tilmanocept has the following structure:

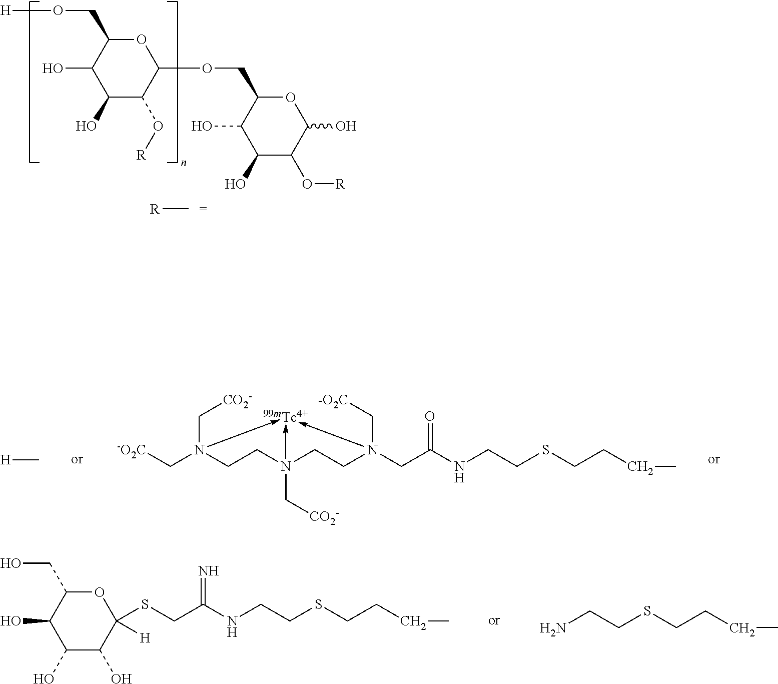

The molecular formula of technetium Tc 99m tilmanocept is [C₆H₁₀O₅]ₙ.(C₁₉H₂₈N₄O₉S^{99m}TC)ₐ.(C₁₃H₂₄N₂O₅S₂)_{b}.(C₅H₁₁NS)_{c}, wherein n is between about 35 and about 58, and n≥(a+b+c). In the commercially marketed version, it contains 3-8 conjugated DTPA (diethylenetriamine pentaacetic acid) moieties (a); 12-20 conjugated mannose moieties (b), and 0-17 unconjugated amine side chains (c).

When used to stage breast cancer and melanoma, technetium Tc 99m labeled tilmanocept (i.e., Lymphoseek) demonstrates rapid clearance from an injection site, rapid and sustained uptake by the sentinel lymph node(s), and low uptake by distal or second-echelon lymph nodes. While the mannose moiety on tilmanocept was known to be responsible for receptor binding, the nature and scope of such binding was not known.

While a variety of devices and techniques may exist for diagnosing and/or treating macrophage related disorders, it is believed that no one prior to the inventor(s) has made or used an invention as described herein.

### SUMMARY

A method of diagnosing an inflammasome-mediated disorder includes administering a pharmaceutical composition to a subject. The composition includes a carrier molecule having a detectable moiety attached thereto. The carrier molecule includes a non-toxic carbohydrate-based backbone. The method also includes after the administering step, detecting a presence of the detectable moiety at a predetermined location in the subject.

### BRIEF DESCRIPTION OF THE DRAWINGS

FIG. 1 is plan views of carrier molecule backbone structures according to exemplary embodiments of the present invention.

### DETAILED DESCRIPTION

The following description of certain examples should not be used to limit the scope of the present invention. Other features, aspects, and advantages of the versions disclosed herein will become apparent to those skilled in the art from the following description. As will be realized, the versions described herein are capable of other different and obvious aspects, all without departing from the invention. Accordingly, the drawings and descriptions should be regarded as illustrative in nature and not restrictive.

The present invention is directed to compositions, methods and kits for the diagnosis and/or treatment of inflammasome-mediated disorders using synthetic macromolecules (e.g., about 2-30 kDa). The inflammasome-mediated disorders may be any disease, disorder or condition in which the inflammasome is activated. Inflammasome-mediated disorders include immune diseases, autoimmune diseases, inflammatory diseases, autoinflammatory diseases, and macrophage-related disorders (i.e., a disease or condition in which macrophages are involved or recruited).

As further discussed below, the compositions described herein include carrier molecules, as well as carrier molecules having one or more detectable moieties and/or therapeutic agents attached thereto. The present invention also provides kits containing such carrier molecules, optionally in a pharmaceutically acceptable carrier (e.g., one which includes a pharmaceutically acceptable vehicle) suitable for administering the carrier molecule to a mammalian subject. In other embodiments, the kit comprises a carrier molecule in a form suitable for labeling with one or more detectable moieties and/or one or more therapeutic agents. In one particular embodiment, the kit comprises the carrier molecule (e.g., a lyophilized powder) in a container along with one or more suitable adjuvants for attaching one or more radioactive isotopes prior to administration. In still further embodiments, diagnostic and/or treatment methods comprising the administration of these carrier molecules to a subject are also provided.

As also used herein, the term “diagnosing” means determining the presence or absence of a medical condition, as well as determining the status of a previously confirmed medical condition in a patient. For example, in the case of cancer, the term diagnosing encompasses determining the presence or absence of cancer, the stage of cancer, and/or the detection of the presence, absence, or stage of a precancerous condition in a patient. Determining the status of a previously confirmed medical condition also includes determining the progress, lack of progress, decline or remission of a medical condition (e.g., a macrophage-related disorder). And the term “treatment” (as well as “treating”) are intended to mean the broadest definition, including not only curing or eliminating a disease, condition or disorder, but also reducing, slowing the progress of, or ameliorating one or more effect of the disease, condition or disorder.

Inflammasome-mediated disorders for which the compositions and methods herein may be used include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behçet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjögren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Applicants have previously discovered that tilmanocept as well as other related carrier molecules described in the '990 patent, as well as other carrier molecules based on a dextran backbone, bind exclusively to the mannose receptor CD206 when administered to mammals. No other receptors bind or transduce tilmanocept and these other carrier molecules, even though there are numerous other mannose receptors found in mammals. CD206 is a C-type lecithin binding protein found on the surface of macrophages. The finding that the CD206 protein on the surface of macrophages is the sole gateway for tilmanocept binding in mammalian patients means that the tilmanocept carrier molecule can be used as the basis for preparing a variety of therapeutically and/or diagnostically effective molecular species for use in the diagnosis and/or treatment of macrophage related disorders.

In the present disclosure, Applicants report their determination that carrier molecules having other monosaccharide-based backbones can be used in place of the dextran backbones described in the '990 patent. Thus, the present invention is directed to compositions, methods and kits for the diagnosis and/or treatment of inflammasome-mediated disorders using synthetic macromolecules comprising a carrier molecule having a carbohydrate-based, non-dextran backbone. In some embodiments, the backbone has a MW of about 1 to about 50 kDa. Unlike the carrier molecules described in the '990 patent, the backbone of the carbohydrate-based carrier molecules described herein comprises a glycan other than dextran, wherein the glycan comprises a plurality of monosaccharide residues (i.e., sugar residues or modified sugar residues). In certain embodiments, the glycan backbone has sufficient monosaccharide residues, as well as optional groups such as one or more amino acids, polypeptides and/or lipids, to provide a MW of about 1 to about 50 kDa.

Embodiments of the backbone include glycans (oligosaccharide or polysaccharide) comprising two or more, three or more, four or more, or five or more monosaccharide residues chosen from the group consisting of mannose, fucose, n-acetylglucosamine, D-galactose, n-acetylgalactoseamine, sialic acid, neuraminic acid and combinations of two or more of the foregoing. In any of these examples, the monosaccharide residues are linear or branched, and in some instances are further conjugated with one or more:
other primary carbohydrates (monosaccharides);
secondary carbohydrates (oligosaccharides);
tertiary carbohydrates (polysaccharides);
quaternary carbohydrates (branched polysaccharides);
an amino acid;
an oligopeptide anchor;
a polypeptide anchor;
a lipid anchor;
a phospholipid anchor;
other fatty anchors; or
combinations of two or more of the foregoing.
   In some embodiments of the foregoing, one or more of the covalent bonds may be altered to be 1→4, 1→6, or alpha or beta.

In still further embodiments, the backbone comprises a glycan (oligosaccharide or polysaccharide) comprising two or more, three or more, four or more, or five or more monosaccharide residues chosen from the group consisting of mannose, glucose, fucose, n-acetylglucosamine, D-galactose, n-acetylgalactoseamine, sialic acid, neuraminic acid, other sugar and modified sugar residues which provide desired targeting specificity, clinical specificity and/or pharmacokinetic characteristics, and combinations of two or more of the foregoing. In any of these examples, the monosaccharide residues are linear or branched, and in some instances are further conjugated with one or more:
other primary carbohydrates (monosaccharides);
secondary carbohydrates (oligosaccharides);
tertiary carbohydrates (polysaccharides);
quaternary carbohydrates (branched polysaccharides);
an amino acid;
an oligopeptide anchor;
a polypeptide anchor;
a lipid anchor;
a phospholipid anchor;
other fatty anchors; or
combinations of two or more of the foregoing.

In further embodiments, the backbone comprises a glycan (oligosaccharide or polysaccharide) comprising two or more, three or more, four or more, or five or more mannose residues. In any of these examples, the mannose residues are, independently, linear or branched (e.g., a first mannose residue having two or three branches off of the first mannose residue). In still further instances the mannose-containing backbone is further conjugated with one or more one or more additional monosaccharide residues chosen from the group consisting of fucose, n-acetylglucosamine, D-galactose, n-acetylgalactoseamine, sialic acid, neuraminic acid and combinations of two or more of the foregoing. In still further examples of mannose-containing backbones, the mannose residues are further conjugated with one or more:
other primary carbohydrates (monosaccharides);
secondary carbohydrates (oligosaccharides);
tertiary carbohydrates (polysaccharides);
quaternary carbohydrates (branched polysaccharides);
an amino acid;
an oligopeptide anchor;
a polypeptide anchor;
a lipid anchor;
a phospholipid anchor;
other fatty anchors; or
combinations of two or more of the foregoing.

In any of the foregoing embodiments wherein the backbone is further conjugated with one or more other primary carbohydrates (monosaccharides), such monosaccharides comprise any of a variety of sugar and modified sugar residues (e.g., sulfated, brominated, or nitrogenated sugar residues), including one or more of: fucose, arabinose, allose, altrose, glucose, galactose, gulose, galactosamine, n-acetylgalactosamine, hammelose, lyxose, levoglucosenone, mannose, mannitol, mannosamine, n-acetylmannosamine, ribose, rhamnose, threose, talose, xylose and combinations of two or more of the foregoing. In certain embodiments, a backbone of compositions herein may comprise a carbohydrate moiety that does not comprise glucose.

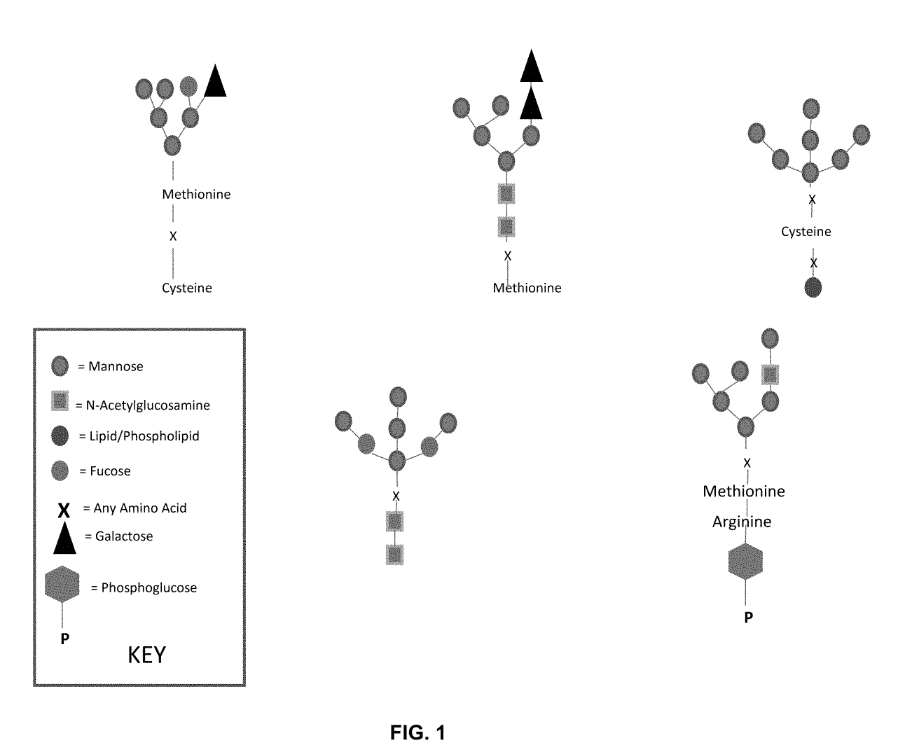

In still further embodiments, the carrier molecule backbone comprises one of the exemplary structures depicted in FIG. 1 hereto. Each of the structures in FIG. 1 comprises a plurality of mannose residues, with additional monosaccharide residues provided before, after or between the mannose residues of the backbone, as shown.

In one particular embodiment, the carrier molecule backbone comprises glucomannan, or a derivative of glucomanan. In another embodiment, the carrier molecule backbone comprises mannan, or a derivative of mannan. In these embodiments, the glucomannan or mannan backbone (or derivatives thereof) may be naturally derived or manufactured synthetically.

The carrier molecules used in the compositions, kits and therapeutic and diagnostic methods described herein are used to deliver a detectable moiety and/or a therapeutic agent (e.g., a cytotoxic agent). The carrier molecules include one or more features which allow a detectable moiety and/or a therapeutic agent to be attached to the molecule, either directly or indirectly (e.g., using a leash). In some embodiments, the carbohydrate-based backbone has a MW of between about 1 and about 50 kDa, while in other embodiments the carbohydrate-based backbone has a MW of between about 5 and about 25 kDa. In still other embodiments, the carbohydrate-based backbone has a MW of between about 8 and about 15 kDa, such as about 10 kDa. While in other embodiments the carbohydrate-based backbone has a MW of between about 1 and about 5 kDa, such as about 2 kDa. The MW of the carbohydrate-based backbone may be selected based upon the inflammasome-mediated disorder, as well as whether the macromolecular construct it to be used for treatment or diagnosis. In addition, unlike the dextran backbone of the '990 patent, the carbohydrate-based backbones described herein do not necessarily need to be cros slink-free, and larger MW backbones (>50 kDa) may even be employed in some instances.

By way of one example, carrier molecules having smaller MW carbohydrate-based backbones may be appropriate for instances where the molecule is desired to cross the blood-brain barrier, or when reduced residence time is desired (i.e., the duration of binding to CD206 is reduced). Carrier molecules having larger MW carbohydrate-based backbones may be appropriate for instances increased residence time is desired (i.e., the duration of binding to CD206 is increased). In still other embodiments, carrier molecules having smaller MW carbohydrate-based backbones (e.g., about 1 to about 5 kDa) may be employed, particularly when the carbohydrate-based backbone is highly branched (e.g., includes one or more highly branched mannose residue and/or includes five or more mannose residues. A branched mannose residue includes, for example, a mannose residue having one more mannose, fucose, n-acetylglucosamine, D-galactose, n-acetylgalactoseamine, sialic acid or neuraminic acid residues attached thereto, either linearly or as one or more additional branches. Such backbones generally will bind to CD206 for longer durations and/or more effectively, thus allowing the use of smaller backbones.

The carrier backbone molecules described herein may be used generally in the same manner as the dextran backbone described in the '990 patent as well in the diagnostic and therapeutic methods and compositions described further herein. However, by proper selection of the monsaccharide residues forming the backbone (e.g., a backbone having two or more mannose residues, and optionally one or more of fucose, n-acetylglucosamine, D-galactose, n-acetylgalactoseamine, sialic acid and neuraminic acid residues), it is generally not necessary to add any additional receptor ligands (i.e., receptor substrates) to the backbone, as described in the '990 patent. The backbone of the carrier molecule binds to the CD206 receptor without the need to add additional receptor substrates via leashes and the like. If desired, however, one or more receptor substrates such as mannose, fucose, n-acetylglucosamine, D-galactose, n-acetylgalactoseamine, sialic acid or neuraminic acid residues may be attached to one or more of the monsaccharide residues of the backbone using leashes, as described in the '990 patent and below with respect to the detectable moieties or therapeutic agents.

The macromolecules used in the therapeutic and diagnostic methods and compositions described herein further include a detectable moiety and/or a therapeutic agent which is attached to the carrier molecule. In some embodiments, the detectable moiety and/or a therapeutic agent is attached directly to the carrier molecule (e.g., via covalent bonding chemistry and synthesis techniques), while in other embodiments they are attached using one or more leashes.

Any of a variety of detectable moieties and/or a therapeutic agents are attached to the carrier molecule, directly or indirectly, for a variety of purposes. As used herein, the term “detectable moiety” means an atom, isotope, or chemical structure which is: (1) capable of attachment to the carrier molecule; (2) non-toxic to humans; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means.

Suitable detectable moieties include, but are not limited to radioisotopes (radionuclides), fluorophores, chemiluminescent agents, bioluminescent agents, magnetic moieties (including paramagnetic moieties), metals (e.g., for use as contrast agents), RFID moieties, enzymatic reactants, colorimetric release agents, dyes, and particulate-forming agents.

By way of specific example, suitable detectable moieties include, but are not limited to:

contrast agents suitable for magnetic resonance imaging (MRI), such as gadolinium (Gd³⁺), paramagnetic and superparamagnetic materials such as superparamagnetic iron oxide;
contrast agents suitable for computed tomographic (CT) imaging, such as iodinated molecules, ytterbium and dysprosium;
radioisotopes suitable for scintigraphic imaging (or scintigraphy) such as technetium-99m, ^{210/212/213/214}Bi, ^{131/140}Ba, _{11/14}C, ⁵¹Cr, ^{67/68}Ga, ¹⁵³Gd, ^{88/90/91}Y, ^{123/124/125/131}I, ^{111/115m}In, ¹⁸F, ¹⁰⁵Rh, ¹⁵³Sm, ⁶⁷Cu, ¹⁶⁶Ho, ¹⁷⁷Lu, ¹⁸⁶Re and ¹⁸⁸Re, ^{32/33}P, ^{46/47}Sc, ^{72/75}Se, ³⁵S, ¹⁸²Ta, ^{123m/127/129/132}Te, ⁶⁵Zn and ^{89/95}Zr;
gamma-emitting agents suitable for single-photon emission computed tomography (SPECT), such as ^{99m}Tc, ¹¹¹In, and ¹²³I.
dyes and fluorescent agents suitable for optical imaging
agents suitable for positron emission tomography (PET) such as ¹⁸F.

The detectable moiety is attached to the carrier molecule in a variety of ways, such as by direct attachment or using a chelator attached to the carrier molecule. In some embodiments, detectable moieties are attached using leashes attached to the carrier backbone. Thereafter, and as described in the '990 patent, a chelator is conjugated to the amino group of one or more leashes and is used to bind the detectable moiety thereto.

As described in the '990 patent, for example, one or more amino-terminated leashes are attached to one or more of the mannose or other monosaccharide residues of the backbone. In some embodiments, the amino-terminated leash(es) comprises —O(CH₂)₃S(CH₂)₂NH₂, wherein a hydroxyl group of the mannose or other monosaccharide moiety is replaced by the amino-terminated leash. This leash may be attached to the backbone by allylating one or more hydroxyl groups on the backbone using allyl bromide. Then, the allyl group(s) is reacted with aminoethanethiol hydrochloride to produce a backbone having one or more —O(CH₂)₃S(CH₂)₂NH₂ leashes.

Various other leashed known to those skilled in the art or subsequently discovered may be used in place of (or in addition to) —O(CH₂)₃S(CH₂)₂NH₂. These include, for example, bifunctional leash groups such as alkylene diamines (H₂N—(CH₂)ᵣ—NH₂), where r is from 2 to 12; aminoalcohols (HO—(CH₂)ᵣ—NH₂), where r is from 2 to 12; aminothiols (HS—(CH₂)ᵣ—NH₂), where r is from 2 to 12; amino acids that are optionally carboxy-protected; ethylene and polyethylene glycols (H—(O—CH₂—CH₂)ₙ—OH, where n is 1-4). Suitable bifunctional diamine compounds include ethylenediamine, 1,3-propanediamine, 1,4-butanediamine, spermidine, 2,4-diaminobutyric acid, lysine, 3,3′-diaminodipropylamine, diaminopropionic acid, N-(2-aminoethyl)-1,3-propanediamine, 2-(4-aminophenyl)ethylamine, and similar compounds. One or more amino acids also can be employed as the bifunctional leash molecule, such as β-alanine, γ-aminobutyric acid or cysteine, or an oligopeptide, such as di- or tri-alanine.

Other bifunctional leashes include:

—NH—(CH₂)ᵣ—NH—, where r is from 2-5,

—O—(CH₂)ᵣ—NH—, where r is from 2-5,

—NH—CH₂—C(O)—,

—O—CH₂CH₂—O—CH₂CH₂—,

—NH—NH—C(O)—CH₂—,

—NH—C(CH₃)₂C(O)—,

—S—(CH₂)ᵣ—C(O)—, where r is from 1-5,

—S—(CH₂)ᵣ—NH—, where r is from 2-5,

—S—(CH₂)ᵣ—O—, where r is from 1-5,

—S(CH₂)—CH(NH₂)—C(O)—,

—S—(CH₂)—CH(COOH)—NH—,

—O—CH₂—CH(OH)—CH₂—S—CH(CO₂H)—NH—,

—O—CH₂—CH(OH)—CH₂—S—CH(NH₂)—C(O)—,

—O—CH₂—CH(OH)—CH₂—S—CH₂—CH₂—NH—,

—S—CH₂—C(O)—NH—CH₂—CH₂—NH—, and

—NH—O—C(O)—CH₂—CH₂—O—P(O₂H)—.

As mentioned previously, one or more detectable moieties are attached to the one or more leashes using a suitable chelator. Suitable chelators include ones known to those skilled in the art or hereafter developed, such as, for example, tetraazacyclododecanetetraacetic acid (DOTA), mercaptoacetylglycylglycyl-glycine (MAG3), diethylenetriamine pentaacetic acid (DTPA), dimercaptosuccinic acid, diphenylethylene diamine, porphyrin, iminodiacetic acid, and ethylenediaminetetraacetic acid (EDTA).

In one particular embodiment, the chelator DTPA is attached to the amino group of one or more leashes conjugated to the carbohydrate-based backbone, and ^{99m}Tc is bound to the DTPA shortly before use. By way of specific example, a lyophilized carbohydrate-based backbone powder having a plurality of leashes and DTPA chelator conjugated thereto is provided in a vial which contains a mixture of 250 mcg of the backbone molecule, 20 mg trehalose dihydrate, 0.5 mg glycine, 0.5 mg sodium ascorbate, and 0.075 mg stannous chloride dihydrate. The contents of the vial are lyophilized and are under nitrogen. Sodium pertechnetate Tc 99m solution is aseptically added to the vial of powder in order to radiolabel the carbohydrate-based backbone powder with Tc 99m. Finally, a sterile, buffered diluent solution comprising 0.04% (w/v) potassium phosphate, 0.11% (w/v) sodium phosphate (heptahydrate), 0.5% (w/v) sodium chloride, and 0.4% (w/v) phenol, with a pH of about 6.8-7.2, is added to the vial. The resulting radiolabeled carbohydrate-based macromolecule is then ready for administration to a patient (e.g., intravenously).

In some embodiments, the carrier molecules used in the therapeutic and diagnostic methods and compositions described herein include a therapeutic agent which is attached to the carrier molecule—either in place of a detectable moiety or in conjunction therewith. As used herein, the term “therapeutic agent” means an atom, isotope, or chemical structure which is effective in curing or eliminating a disease or other condition, as well those which are effective in reducing, slowing the progress of, or ameliorating the adverse effects of a disease or other condition. Therapeutic agents include cytotoxic agents.

In some embodiments, the therapeutic agent comprises a high energy killing isotope which has the ability to kill macrophages and tissue in the surrounding macrophage environment. Suitable radioisotopes include: ^{210/212/213/214}Bi, ^{131/140}Ba, _{11/14}C, ⁵¹Cr, ^{67/68}Ga, ¹⁵³Gd, ^{99m}Tc, ^{88/90/91}Y, ^{123/124/125/131}I, ^{111/115m}In, ¹⁸F, ¹⁰⁵Rh, ¹⁵³Sm, ⁶⁷Cu, ¹⁶⁶Ho, ¹⁷⁷Lu, ¹⁸⁶Re and ¹⁸⁸Re, ^{32/33}P, ^{46/47}Sc, ^{72/75}Se, ³⁵S, ¹⁸²Ta, ^{123m/127/129/132}Te, ⁶⁵Zn and ^{89/95}Zr.

In other embodiments, the therapeutic agent comprises a non-radioactive species selected from, but not limited to, the group consisting of: Bi, Ba, Mg, Ni, Au, Ag, V, Co, Pt, W, Ti, Al, Si, Os, Sn, Br, Mn, Mo, Li, Sb, F, Cr, Ga, Gd, I, Rh, Cu, Fe, P, Se, S, Zn and Zr.

In still further embodiments, the therapeutic agent is selected from the group consisting of cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radiosensitizing drugs. By way of more specific example, the therapeutic agent is selected from the group consisting of mechlorethamine, triethylenephosphoramide, cyclophosphamide, ifosfamide, chlorambucil, busulfan, melphalan, triaziquone, nitrosourea compounds, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, and dynemicins.

In embodiments wherein the therapeutic agent is a hormone or hormone antagonist, the therapeutic agent may be selected from the group consisting of prednisone, hydroxyprogesterone, medroprogesterone, diethylstilbestrol, tamoxifen, testosterone, and aminogluthetimide.

In embodiments wherein the therapeutic agent is a prodrug, the therapeutic agent may be selected from the group consisting of phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that can be converted to the more active cytotoxic free drug.

The therapeutic agent is attached to the carrier molecule in a variety of ways. In some embodiments, one or more leashes are conjugated to the backbone molecule, and a chelator is conjugated to the leashes (e.g., to the amino group of amino-terminated leashes). The chelator is used to bind the therapeutic agent thereto. Suitable chelators include ones known to those skilled in the art or hereafter developed, such as, for example, tetraazacyclododecanetetraacetic acid (DOTA), mercaptoacetylglycylglycyl-glycine (MAG3), diethylenetriamine pentaacetic acid (DTPA), dimercaptosuccinic acid, diphenylehtylene diamine, porphyrin, iminodiacetic acid, and ethylenediaminetetraacetic acid (EDTA).

The macromolecular compounds described herein may be administered in a variety of ways, using any of a variety of pharmaceutically acceptable carriers and vehicles. For example, a pharmaceutical preparation comprising the carrier molecule having one or more detectable moieties and/or therapeutic agents attached thereto, in combination with a pharmaceutically acceptable carrier is administered via intravenous injection, subcutaneous injection, intradermal injection, parenchymal introduction, inhalation, pulmonary lavage, suppository, or oral, sublingual, intracranial, intraocular, intranasal, or intraaural introduction.

In a further specific embodiment for diagnosing and/or treating tuberculosis, the detectable moiety comprises ⁶⁸Ga, and the therapeutic agent comprises ⁶⁸Ga and/or Ga. In still further embodiments, a composition for both diagnosing and treating tuberculosis is provided, wherein the both ⁶⁸Ga and Ga (i.e., non-radioactive Ga) are conjugated to the carrier molecule.

While several compositions and methods for the diagnosis and/or treatment of macrophage-related disorders have been discussed in detail above, it should be understood that the compositions, features, configurations, and methods of using the compositions discussed are not limited to the contexts provided above.

1. A method of diagnosing an inflammasome-mediated disorder comprising the steps of:
a. administering a pharmaceutical composition to a subject, said composition including a carrier molecule having a detectable moiety attached thereto, said carrier molecule comprising a non-toxic carbohydrate-based backbone; and
b. after said administering step, detecting the presence of said detectable moiety at a predetermined location in the subject.

2. The method of claim 1, wherein said carbohydrate-based backbone comprises a mannose-containing glycan.

3. The method of claim 2, wherein said mannose-containing glycan comprises an oligosaccharide having two or more mannose residues.

4. The method of claim 2, wherein said mannose-containing glycan comprises a polysaccharide having two or more mannose residues.

5. The method of claim 1, wherein the backbone is further conjugated with one or more:
other primary carbohydrates (monosaccharides);
secondary carbohydrates (oligosaccharides);
tertiary carbohydrates (polysaccharides);
quaternary carbohydrates (branched polysaccharides);
an amino acid;
an oligopeptide anchor;
a polypeptide anchor;
a lipid anchor;
a phospholipid anchor;
other fatty anchors; and
combinations of two or more of the foregoing.

6. The method of claim 1, wherein said backbone comprises a mannose-containing glycan, and includes one or more additional monosaccharide residues chosen from the group consisting of fucose, n-acetylglucosamine, D-galactose, n-acetylgalactoseamine, sialic acid and neuraminic acid.

7. The method of claim 1, wherein said carrier molecule has at least one leash wherein said detectable moiety is attached to the backbone via said leash.

8. The method of claim 7, wherein said leash is —O(CH₂)₃S(CH₂)₂NH₂.

9. The method of claim 1, wherein said detecting step comprises detecting the presence of the detectable moiety in tissue.

10. The method of claim 1, wherein said detecting step comprises performing sentinel node imaging on the subject.

11. The method of claim 1, wherein the inflammasome-mediated disorder is an angiogenic disorder.

12. The method of claim 1, wherein the inflammasome-mediated disorder is cancer, tuberculosis, HIV, or multiple sclerosis.

13. A method of treating an inflammasome-mediated disorder comprising the step of administering a pharmaceutical composition to a subject, said composition including a carrier molecule having a therapeutic agent attached thereto, said carrier molecule comprising a non-toxic carbohydrate-based backbone.

14. The method of claim 13, wherein said carbohydrate-based backbone comprises a mannose-containing glycan.

15. The method of claim 14, wherein said mannose-containing glycan comprises an oligosaccharide having two or more mannose residues.

16. The method of claim 14, wherein said mannose-containing glycan comprises a polysaccharide having two or more mannose residues.

17. The method of claim 14, wherein the mannose-containing backbone is further conjugated with one or more:
other primary carbohydrates (monosaccharides);
secondary carbohydrates (oligosaccharides);
tertiary carbohydrates (polysaccharides);
quaternary carbohydrates (branched polysaccharides);
an amino acid;
an oligopeptide anchor;
a polypeptide anchor;
a lipid anchor;
a phospholipid anchor;
other fatty anchors; and
combinations of two or more of the foregoing.

18. The method of claim 13, wherein said backbone includes one or more additional monosaccharide residues chosen from the group consisting of fucose, n-acetylglucosamine, D-galactose, n-acetylgalactoseamine, sialic acid and neuraminic acid.

19. The method of claim 13, wherein said carrier molecule has at least one leash wherein said therapeutic agent is attached to the backbone via said leash.

20. The method of claim 19, wherein said leash is —O(CH₂)₃S(CH₂)₂NH₂.

21. The method of claim 13, wherein the inflammasome-mediated disorder is an inflammatory disorder.

22. The method of claim 13, wherein the inflammasome-mediated disorder is an angiogenic disorder.

23. The method of claim 13, wherein the inflammasome-mediated disorder is cancer, tuberculosis, HIV, or multiple sclerosis.

24. A method of diagnosing and/or treating tuberculosis comprising the steps of:
a. administering a pharmaceutical composition to a subject, said composition comprising including a non-toxic carbohydrate-based backbone having a detectable moiety and/or therapeutic agent attached thereto; and
b. for a diagnostic procedure, after said administering step, detecting the presence of said radioactive isotope in the subject's lung tissue.

25. The method of claim 24, wherein said detectable moiety and/or therapeutic agent comprises ⁶⁸Ga and/or Ga.

26. A composition according to claim 1.

27. A kit for the preparation of a diagnostic and/or therapeutic composition according to claim 1.
